SEQUENCE LISTING (3 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 21 aa, Artificial Sequence
  Synthetic
SFSQNSRHPSQNPPVLKRHQR

SEQ ID NO: 3 - protein, 1445 aa, Artificial Sequence
  Synthetic
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKCVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKCEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2016/0264645 A1
(43) Pub. Date: Sep. 15, 2016

(54) Stabilized Factor VIII Variants

(71) Applicant: Novo Nordisk A/S, Bagsvaerd (DK)

(72) Inventors: Henrik Oestergaard, Oelstykke (DK); Marianne Kjalke, Frederikssund (DK); Ole Hvilsted Olsen, Broenshoej (DK); Lars Thim, Gentofte (DK); Henning Ralf Stennicke, Kokkedal (DK)

(21) Appl. No.: 15/170,502

(22) Filed: Jun. 1, 2016

Related U.S. Application Data
(63) Continuation of application No. 13/808,204, filed on Mar. 7, 2013.
(60) Provisional application No. 61/365,478, filed on Jul. 19, 2010.

(30) Foreign Application Priority Data
Jul. 15, 2010 (EP) 10169592.2

(51) Int. Cl. C07K 14/755; A61K 47/48; A61K 38/37

(52) U.S. Cl. CPC C07K 14/755; A61K 38/37; A61K 47/48215; A61K 47/4823

(57) ABSTRACT

The present invention relates to modified coagulation factors. In particular, the present invention relates to stabilized Factor VIII molecules conjugated with a half life extending moiety as well as use of such molecules.

### CROSS-REFERENCE TO RELATED APPLICATIONS

This application is a continuation of U.S. application Ser. No. 13/808,204, filed Mar. 7, 2013 (pending), which is a 35 U.S.C. §371 National Stage application of International Application PCT/EP2011/061349 (WO 2012/007324), filed Jul. 6, 2011, which claimed priority of European Patent Application 10169592.2, filed Jul. 15, 2010; this application claims priority under 35 U.S.C. §119 of U.S. Provisional Application 61/365,478; filed Jul. 19, 2010; the contents of which are incorporated herein by reference.

### SEQUENCE LISTING

The instant application contains a Sequence Listing which has been submitted in ASCII format via EFS-Web and is hereby incorporated by reference in its entirety. Said ASCII copy, created on May 31, 2015, is named 8188US03SeqList.txt and is 33,883 bytes in size.

### FIELD OF THE INVENTION

The present invention relates to modified coagulation factors. In particular, the present invention relates to stabilized Factor VIII molecules conjugated to a half life extending moiety. The invention furthermore relates to use of such molecules.

### BACKGROUND OF THE INVENTION

Haemophilia A is an inherited bleeding disorder caused by deficiency or dysfunction of coagulation factor VIII (FVIII) activity. The clinical manifestation is not on primary haemostasis—formation of the blood clot occurs normally—but the clot is unstable due to a lack of secondary thrombin formation. The disease is treated by intravenous injection of coagulation factor FVIII which is either isolated from blood or produced recombinantly. Current treatment recommendations are moving from traditional on-demand treatment towards prophylaxis. The circulatory half life of endogenous FVIII bound to von Willebrandt Factor is 12-14 hours and prophylactic treatment is thus to be performed several times a week in order to obtain a virtually symptom-free life for the patients. IV administration is for many, especially children and young persons, associated with significant inconvenience and/or pain.

Various methods have been employed in the development of a Factor VIII variant with significantly prolonged circulatory half life. A number of these methods relate to conjugation of Factor VIII with hydrophilic polymers such as e.g. PEG (poly ethylene glycol). WO03031464 discloses an enzymatic approach where PEG groups can be attached to glycans present on the polypeptide. Blood-2009-11-254755 discloses introduction of surface exposed Cys-residues to which PEG groups can be specifically conjugated.

Introduction of disulfide bridges to the FVIII molecule is known from WO02103024. However, such FVIII variants comprising a disulfide bridge did, however, not result in a prolonged in vivo circulatory half life.

There is thus a need in the art for FVIII variants with factor VIII activity and a significantly prolonged in vivo ciruculatory half life.

### SUMMARY OF THE INVENTION

The present invention relates to a recombinant FVIII variant having FVIII activity, wherein the FVIII variant is conjugated with a half life extending moiety, and wherein amino acid alterations have been introduced in the FVIII variant in order to increase the in vitro stability of the variant. The present invention furthermore relates to use of such molecules in therapy. The molecules according to the invention have a significantly increased in vivo circulatory half life as compared to wt Factor VIII.

### BRIEF DESCRIPTION OF DRAWINGS

FIG. 1. Maximum level of thrombin activity obtained at the different concentration of wild type FVIII and variants. Data are mean and SEM of data from 5 individual experiments each normalized to the rate obtained by 2.7 nM wild type FVIII.

### DESCRIPTION OF THE INVENTION

### Definitions

Von Willebrandt Factor (vWF): vWF is a large mono-/multimeric glycoprotein present in blood plasma and produced constitutively in endothelium (in the Weibel-Palade bodies), megakaryocytes (α-granules of platelets), and subendothelial connective tissue. Its primary function is binding to other proteins, particularly Factor VIII and it is important in platelet adhesion to wound sites. Factor VIII is bound to vWF while inactive in circulation; Factor VIII degrades rapidly or is cleared when not bound to vWF. It thus follows that reduction or abolishment of vWF binding capacity in FVIII would be considered as a highly undesirable approach in obtaining Factor FVIII variants with prolonged circulatory half life. It may however be possible to reduce or abolish vWF by site directed mutagenesis if the molecule is conjugated to a half life extending moiety. The region in Factor VIII responsible for binding to vWF is the region spanning residues 1670-1684 as disclosed in EP0319315. It is envisaged that Factor VIII point and/or deletion mutants involving this area will modify the ability to bind to vWF. Examples of particularly preferred point mutations according to the present invention include variants comprising one or more of the following point mutations: Y1680F, Y1680R, Y1680N, and E1682T, and Y1680C.

Factor VIII molecules: FVIII/Factor VIII is a large, complex glycoprotein that primarily is produced by hepatocytes. FVIII consists of 2351 amino acids, including signal peptide, and contains several distinct domains, as defined by homology. There are three A-domains, a unique B-domain, and two C-domains. The domain order can be listed as NH2-A1-A2-B-A3-C1-C2-COOH. FVIII circulates in plasma as two chains, separated at the B-A3 border. The chains are connected by bivalent metal ion-bindings. The A1-A2-B chain is termed the heavy chain (HC) while the A3-C1-C2 is termed the light chain (LC). “Factor VIII” or “FVIII” as used herein refers to a human plasma glycoprotein that is a member of the intrinsic coagulation pathway and is essential to blood coagulation. “Native FVIII” is the full length human FVIII molecule as shown in SEQ ID NO: 1 (amino acid 1-2332). The B-domain is spanning amino acids 741-1648 in SEQ ID NO: 1.

(wt human FVIII):
SEQ ID NO: 1
ATRRYYLGAVELSWDYMQSDLGELPVDARFPPRVPKS-
FPFNTSVVYKKTLFVEFTDHLFNIAKPRPPVVMGLLGPTIQAEVYDTVVITLKNMASH-
PVSLHAVGVSYWKASEGAEYDDQTSQREKEDDKVFPGG-
SHTYVWQVLKENGPMASDPLCLTYSYLSHVDLVKDLNSGLIGALLVCREGSLAKEKTQTLHK-
FILLFAVFDEGKSWHSETKNSLMQDRDAASARAWPKMHTVNGYVNRSLPGLIGCHRKSVY-
WHVIGMGTTPEVHSIFLEGHTFLVRNHRQASLEISPITFLTAQ-
TLLMDLGQFLLFCHISSHQHDGMEAYVKVDSCPEEPQLRMKNNEEAEDYDDDLT-
DSEMDVVRFDDDNSPSFIQIRSVAKKHPKTVVVHYIAAEEEDWDYAPLVLAPDDRSYKSQYLN-
NGPQRIGRKYKKVRFMAYTDETFKTREAIQHESGILGPLLYGEVGDTLLIIFKNQASRPYNI-
YPHGITDVRPLYSRRLPKGVKHLKDFPILPGEIFKYKVVTVTVEDGPTKSDPRCLTRYYSS-
FVNMERDLASGLIGPLLICYKESVDQRGNQIMSDKRNVILFSVFDENRSVVYLTENI-
QRFLPNPAGVQLEDPEFQASNIMHSINGYVFDSLQLSVCLHEVAYVVYILSIGAQTD-
FLSVFFSGYTFKHKMVYEDTLTLFPFSGETVFMSMENPGLWILGCHNSDFRNRG-
MTALLKVSSCDKNTGDYYEDSYEDISAYLLSKNNAIEPRSFSQNSRHPSTRQKQFNATTIPEN-
DIEKTDPWFAHRTPMPKIQNVSSSDLLMLLRQSPTPHGLSLSDLQEAKYETFSDDP-
SPGAIDSNNSLSEMTHFRPQLHHSGDMVFTPESGLQLRLNEKLGTTAATELKKLD-
FKVSSTSNNLISTIPSDNLAAGTDNTSSLGPPSMPVHYDSQLDTTLFGKKSSPLTESGG-
PLSLSEENNDSKLLESGLMNSQESS-
WGKNVSSTESGRLFKGKRAHGPALLTKDNALFKVSISLLKTNKTSNNSATNRKTHIDGPSLLIEN
SPSVWQNILESDTEFKKVTPLIHDRMLMDKNATALRLNHMSNKTTSSKNMEMVQQKKEG-
PIPPDAQNPDMSFFKMLFLPESARWIQRTHGKNSLNSGQGP-
SPKQLVSLGPEKSVEGQNFLSEKNKVVVGKGEFTKDVGLKEMVFPSSRNLFLTNLD-
NLHENNTHNQEKKIQEEIEKKETLIQENVVLPQIHTVTGTKNFMKNLFLLSTRQNVEGSYDGA-
YAPVLQDFRSLNDSTNRTKKHTAHFSKKGEEENLEGLGNQTKQIVEKYACTTRISP-
NTSQQNFVTQRSKRALKQFRLPLEETELEKRIIVDDTSTQWSKNMKHLTPSTLTQI-
DYNEKEKGAITQSPLSDCLTRSHSIPQANRSPLPIAKVSS-
FPSIRPIYLTRVLFQDNSSHLPAASYRK-
KDSGVQESSHFLQGAKKNNLSLAILTLEMTGDQREVGSLGTSATNSVTYKKVENTVLPKPDLP
KTSGKVELLPKVHIYQKDLFPTETSNGSPGHLDLVEGSLLQGTEGAIK-
WNEANRPGKVPFLRVATESSAKTPSKLLDPLAWDNHYGTQIPKEEWKSQEKSPEKTAF-
KKKDTILSLNACESNHAIAAINEGQNKPEIEVTWAKQGRTERLCSQNPPVLKRHQREITRT-
TLQSDQEEIDYDDTISVEMKKEDFDIYDEDENQSPRSFQKKTRHYFI-
AAVERLWDYGMSSSPHVLRNRAQSGSVPQFKKVVFQEFTDGSFTQPLYRGELNEH-
LGLLGPYIRAEVEDNIMVTFRNQASRPYSFYSSLISYEEDQRQGAEPRKNFVKPNETK-
TYFWKVQHHMAPTKDEFDCKAWAYFSDVDLEKDVHSGLIG-
PLLVCHTNTLNPAHGRQVTVQEFALFFTIFDETKSVVYFTENMERN-
CRAPCNIQMEDPTFKENYRFHAINGYIMDTLPGLVMAQDQRIRVVYLLSMGSNE-
NIHSIHFSGHVFTVRKKEEYKMALYNLYPGVFETVEM-
LPSKAGIWRVECLIGEHLHAGMSTLFLVYSNKCQTPLGMASGHIRDFQITASGQYGQWAP-
KLARLHYSGSINAWSTKEPFSWIKVDLLAPMIIHGIKTQGARQKFSSLYISQFIIMYSLD-
GKKWQTYRGNSTGTLMVFFGNVDSSGIKHNIFNPPIIARYIRLHPTHYSIRSTLR-
MELMGCDLNSCSMPLGMESKAISDAQITASSYFTNMFAT-
WSPSKARLHLQGRSNAWRPQVNNPKEWLQVDFQKTMKVTGVTTQGVKSLLTSMYVKE-
FLISSSQDGHQWTLFFQNGKVKVFQGNQDSFTPVVNSLDPPLLTRYLRI-
HPQSVVVHQIALRMEVLGCEAQDLY

The FVIII molecules/variants according to the present invention may be B domain truncated Factor FVIII molecules wherein the remaining domains correspond closely to the sequence as set forth in amino acid no 1-740 and 1649-2332 in SEQ ID NO. 1.

FVIII variants according to the invention may differ slight from the sequence set forth in SEQ ID NO: 1, meaning that the three A-domains and the two C-domains may differ slightly e.g. 1, 2, 3, 4, 5, 6, 7, 8, 9, 10, 11, 12, 13, 14, 15, 16, 17, 18, 19, or 20 amino acids from the amino acid sequence as set forth in SEQ ID NO: 1 (amino acids 1-740 and 1649-2332) due to the fact that amino acid substitutions are introduced in order to increase in vitro stability. Other mutations may be introduced in order to e.g. reduce vWF binding capacity. Furthermore, it is plausible that amino acid modifications (substitutions, deletions, etc.) are introduced other places in the molecule in order to modify the binding capacity of Factor VIII with various other components such as e.g. LRP, various receptors, other coagulation factors, cell surfaces, introduction and/or abolishment of glycosylation sites, etc.

Factor VIII variants according to the present invention have Factor VIII activity, meaning the ability to function in the coagulation cascade in a manner functionally similar or equivalent to FVIII, induce the formation of FXa via interaction with FIXa on an activated platelet, and support the formation of a blood clot. The activity can be assessed in vitro by techniques well known in the art such as e.g. clot analysis, endogenous thrombin potential analysis, etc. Factor VIII molecules according to the present invention have FVIII activity being at least about 10%, at least 20%, at least 30%, at least 40%, at least 50%, at least 60%, at least 70%, at least 80%, at least 90%, and 100% or even more than 100% of that of native human FVIII.

Intrinsic Stability/In Vitro Stability of FVIII:

The “intrinsic stability” or the “in vitro stability” of a polypeptide such as e.g FVIII may sometimes be referred to as the “stability”, the “physical stability”, the “inherent stability”, the “structural stability”, the “chemical stability”, “intrinsic stability”, the “thermodynamic stability”, the “thermal stability”, the “folding stability” etc. The common theme for such terms is that they refer to the in vitro stability of the polypeptide and this in vitro stability can be seen as the sum of the inherent properties of the polypeptide that act to stabilize its three dimensional structure. There are significant differences between FVIII in vivo stability and FVIII in vitro stability because FVIII is subject to a large number of clearance mechanisms in vivo. It has thus far not been considered to obtain a prolonged in vivo circulatory half life of FVIII by improving the in vitro stability of the molecule.

Conjugation of FVIII with various side chains is known in the art as a mean for obtaining a prolonged in vivo circulatory half life of FVIII. It has previously been demonstrated that circulatory half-life can be increased approximately 2-fold, i.e., to about 24 hours, by e.g. conjugation of the FVIII molecule. The in vitro stability of wt FVIII, as determined by a half-life in TAP/hirudin anti-coagulated plasma at 37° C. is about 30 hours.

Without being bound by theory, the rationale behind the present invention is that the in vitro stability of FVIII becomes the limiting parameter for any further prolongation of the in vivo circulatory half life once the molecule has been conjugated with one or more side chains. The inventors of the present invention have thus shown that there is a surprisingly enhanced effect in the combination of one or more covalently linked side chains combined with FVIII point mutations/amino acid alterations that result in increased in vitro stability of the FVIII molecule, An additional surprising effect that may be obtained with molecules according to the present invention is that the resulting FVIII variants may furthermore possess a significantly increased specific activity resulting in a more potent molecule as a result of particular mutations/amino acid alterations that lead to a decreased rate of dissociation of the A2 domain from the activated FVIII molecule. The guadinium chloride assay disclosed in Example 6 may e.g. be used for determining if FVIII variants have increased in vitro stability compared to e.g. wt. FVIII or B domain truncated FVIII variants without any in vitro stabilizing amino acid alterations.

Amino acid alterations as used herein refer to amino acid substitutions, deletions, and additions. Preferably, amino acid alterations according to the present invention are in the form of one, two, three or a few (e.g. 1, 2, 3, 4, 5, 6, 7, 8, 9, 10, 11, 12, 13, 14, or 15) amino acid substitutions within one or more of the A1, A2, A3, C1 and C2 domains. Numerous ways of obtaining a FVIII variant with increased in vitro stability can be envisaged such as e.g. introduction of one, two or three disulfide bridges and/or introduction of hydrophobic amino acid residues, and/or introduction of electrostatic interactions, and/or introduction of amino acid substitutions which stabilize the binding of metal ions bound to FVIII, e.g. Cu or Zn, and/or introduction of amino acid substitutions which result in increased resistance to oxidation.

B domain truncated/deleted Factor VIII molecule: The B-domain in Factor VIII spans amino acids 741-1648 in SEQ ID NO: 1. The B-domain is cleaved at several different sites, generating large heterogeneity in circulating plasma FVIII molecules. The exact function of the heavily glycosylated B-domain is unknown. What is known is that the domain is dispensable for FVIII activity in the coagulation cascade. Recombinant FVIII is thus frequently produced in the form of B domain deleted/truncated variants.

Endogenous full length FVIII is synthesized as a single-chain precursor molecule. Prior to secretion, the precursor is cleaved into the heavy chain and the light chain. Recombinant B domain-deleted FVIII can be produced from two different strategies. Either the heavy chain without the B-domain and the light chain are synthesized individually as two different polypeptide chains (two-chain strategy) or the B-domain deleted FVIII is synthesized as a single precursor polypeptide chain (single-chain strategy) that is cleaved into the heavy and light chains in the same way as the full-length FVIII precursor.

In a B domain-deleted/truncated FVIII precursor polypeptide, the heavy and light chain moieties are normally separated by a linker. To minimize the risk of introducing immunogenic epitopes in the B domain-deleted FVIII, the sequence of the linker is preferable derived from the FVIII B-domain. As a minimum, the linker must comprise a recognition site for the protease that cleaves the B domain-deleted FVIII precursor polypeptide into the heavy and light chain. In the B domain of full length FVIII, amino acid 1644-1648 constitutes this recognition site. The thrombin site leading to removal of the linker on activation of B domain-deleted FVIII is located in the heavy chain. Thus, the size and amino acid sequence of the linker is unlikely to influence its removal from the remaining FVIII molecule by thrombin activation. Deletion/truncation of the B domain is an advantage for production of FVIII. Nevertheless, parts of the B domain can be included in the linker without reducing the productivity. The negative effect of the B domain on productivity has not been attributed to any specific size or sequence of the B domain.

According to a preferred embodiment, the truncated/deleted B domain comprises only one potential O-glycosylation sites and one or more side groups/half life extending moieties are covalently conjugated to this O-glycosylation site, preferably via a linker.

The O-linked oligosaccharides in the B-domain truncated molecules according to the invention may be attached to O-glycosylation sites that were either artificially created by recombinant means and/or by generation of new O-glycosylation sites by truncation of the B-domain. In both cases, such molecules may be made by designing a B-domain trunctated Factor VIII amino acid sequence and subsequently subjecting the amino acid sequence to an in silico analysis predicting the probability of O-glycosylation sites in the truncated B-domain. Molecules with a relatively high probability of having such glycosylation sites can be synthesized in a suitable host cell followed by analysis of the glycosylation pattern and subsequent selection of molecules having O-linked glycosylation in the truncated B-domain. The Factor VIII molecule also contains a number of N-linked oligosaccharides and each of these may potentially serve as an anchor for attachment of a half life extending moiety.

The length of the B domain in the wt FVIII molecule is about 907 amino acids. The length of the truncated B domain in FVIII variants according to the present invention may vary from about 10 to about 800 amino acids, such as e.g. from about 10 amino acids to about 700 acids, such as e.g. about 12-500 amino acids, 12-400 amino acids, 12-300 amino acids, 12-200 amino acids, 15-100 amino acids, 15-75 amino acids, 15-50 amino acids, 15-45 amino acids, 20-45 amino acids, 20-40 amino acids, or 20-30 amino acids. The truncated B-domain may comprise fragments of the heavy chain and/or the light chain and/or an artificially introduced sequence that is not found in the wt FVIII molecule. The terms “B-domain truncated” and “B-domain deleted” may be used interchangeably herein.

Half life extending moiety/Side chain/side group: FVIII variants according to the present invention are covalently conjugated with a half life extending moiety/side group either via post-translational modification or in the form of a fusion protein. One or more of the following modifications of FVIII may thus be carried out: alkylation, acylation, ester formation, di-sulfide or amide formation or the like. This includes PEGylated FVIII, cysteine-PEGylated FVIII and variants thereof. The FVIII variants according to the invention may also be conjugated to other biocompatible fatty acids and derivatives thereof, hydrophilic polymers (Hydroxy Ethyl Starch, Poly Ethylen Glycol, hyaluronic acid, heparosan polymers, Phosphorylcholine-based polymers, fleximers, dextran, poly-sialic acids), polypeptides (antibodies, antigen binding fragments of antibodies, Fc domains, transferrin, albumin, Elastin like peptides (MacEwan S R, Chilkoti A. Biopolymers. 2010; 94:60), XTEN polymers (Schellenberger V et al. Nat Biotechnol. 2009; 27:1186), PASylation or HAPylation (Schlapschy M et al. Protein Eng Des Sel. 2007; 20:273), Albumin binding peptides (Dennis M S et al. J Biol Chem. 2002, 277:35035)) etc.

FVIII according to the present invention may be acylated by one or more hydrophobic half life extending moities/side groups—optionally via a linker. Compounds having a —(CH₂)₁₂-moiety are possible albumin binders in the context of the present invention. Hydrophobic half life extending moieties may sometimes be referred to as “albumin binders” due to the fact that such moieties be capable of forming non-covalent complexes with albumin, thereby promoting the circulation of the acylated FVIII variant in the blood stream, due to the fact that the complexes of the acylated FVIII variant and albumin is only slowly disintegrated to release the FVIII variant. FVIII can be acylated using chemical methods as well as enzymatic “glyco-acylation” methods essentially following the processes as disclosed in WO03031464. Enzymatic methods have the advantages of avoiding use of any organic solvents.

The term “PEGylated FVIII” means FVIII, conjugated with a PEG molecule. It is to be understood, that the PEG molecule may be attached to any part of FVIII including any amino acid residue or carbohydrate moiety. The term “cysteine-PEGylated FVIII” means FVIII having a PEG molecule conjugated to a sulfhydryl group of a cysteine introduced in FVIII.

PEG is a suitable polymer molecule, since it has only few reactive groups capable of cross-linking compared to polysaccharides such as dextran. In particular, monofunctional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule are provided in activated form, i.e. with reactive functional groups. The PEGylation may be directed towards conjugation to all available attachment groups on the polypeptide (i.e. such attachment groups that are exposed at the surface of the polypeptide) or may be directed towards one or more specific attachment groups, e.g. the N-terminal amino group (U.S. Pat. No. 5,985,265), N- and/or O-linked glycans, etc. Furthermore, the conjugation may be achieved in one step or in a stepwise manner (e.g. as described in WO 99/55377). An enzymatic approach for coupling half life extending moieties to 0- and/or N-linked glycans is disclosed in WO03031464.

Fusion protein: Fusion proteins/chimeric proteins, are proteins created through the joining of two or more genes which originally coded for separate proteins. Translation of this fusion gene results in a single polypeptide with functional properties derived from each of the original proteins. The side chain of the FVIII variants according to the present invention may thus be in the form of a polypeptide fused to FVIII. FVIII according to the present invention may thus be fused to peptides that can confer a prolonged half life to the FVIII such as e.g. antibodies and “Fc fusion derivatives” or “Fc fusion proteins”.

Fc fusion protein is herein meant to encompass FVIII fused to an Fc domain that can be derived from any antibody isotype, although an IgG Fc domain will often be preferred due to the relatively long circulatory half life of IgG antibodies. The Fc domain may furthermore be modified in order to modulate certain effector functions such as e.g. complement binding and/or binding to certain Fc receptors. Fusion of FVIII with an Fc domain, having the capacity to bind to FcRn receptors, will generally result in a prolonged circulatory half life of the fusion protein compared to the half life of the wt FVIII protein. Mutations in positions 234, 235 and 237 in an IgG Fc domain will generally result in reduced binding to the FcγRI receptor and possibly also the FcγRIIa and the FcγRIII receptors. These mutations do not alter binding to the FcRn receptor, which promotes a long circulatory half life by an endocytic recycling pathway. Preferably, a modified IgG Fc domain of a fusion protein according to the invention comprises one or more of the following mutations that will result in decreased affinity to certain Fc receptors (L234A, L235E, and G237A) and in reduced C1q-mediated complement fixation (A330S and P331S), respectively.

FVIII may also be fused to any other polypeptide having the ability to confer a prolonged circulatory half life to FVIII, such as e.g. proteins with the capacity to bind specifically to platelets, such as e.g. antibodies specific for proteins expressed on the surface of platelets (e.g. AP3 antibodies).

FVIII may also be fused to “polypeptide extensions”, such as e.g.: HAPylation (Glyₓ-Ser_{y})ₙ (Protein Eng Des Sel. 2007 June; 20(6):273-84), XTEN/rPEG (poly non-hydrophobic amino acids) (Nat Biotechnol. 2009 December; 27(12):1186-90), PASylation (fusion with inert and degradable moities composed of the amino acids Pro, Ala, and Ser provides an efficient way to confer a large hydrodynamic volume to a biologically active protein, thus retarding its clearance via kidney filtration), ELP (Elastin Lilke Peptide) (Biopolymers. 2010; 94(1):60-77), and albumin binding peptides (J Biol Chem. 2002 Sep. 20; 277(38):35035-43).

Glycoprotein: The term “glycoprotein” is intended to encompass peptides, oligopeptides and polypeptides containing one or more oligosaccharides (glycans) attached to one or more amino acid residues of the “back bone” amino acid sequence. The glycans may be N-linked or O-linked.

The term “terminal sialic acid” or, interchangeable, “terminal neuraminic acid” is thus intended to encompass sialic acid residues linked as the terminal sugar residue in a glycan, or oligosaccharide chain, i.e., the terminal sugar of each antenna is N-acetylneuraminic acid linked to galactose via an α2->3 or α2->6 linkage.

The term “galactose or derivative thereof” means a galactose residue, such as natural D-galactose or a derivative thereof, such as an N-acetylgalactosamine residue.

The term “terminal galactose or derivative thereof” means the galactose or derivative thereof linked as the terminal sugar residue in a glycan, or oligosaccharide chain, e.g., the terminal sugar of each antenna is galactose or N-acetylgalactosamine.

The term “asialo glycoprotein” is intended to include glycoproteins wherein one or more terminal sialic acid residues have been removed, e.g., by treatment with a sialidase or by chemical treatment, exposing at least one galactose or N-acetylgalactosamine residue from the underlying “layer” of galactose or N-acetylgalactosamine (“exposed galactose residue”).

In general, N-linked glycans which are not part of wild type FVIII can be introduced into the FVIII molecules of the invention, by introducing amino acid mutations so as to obtain N-X-S/T motifs. The FVIII molecules of the present invention contain 1, 2, 3, 4, 5, 6, 7, 8, 9, or 10, or more, N-linked glycans. The structure of N-linked glycans are of the high-mannose or complex form. High mannose glycans contain terminal mannose residues at the non-reducing end of the glycan. Complex N-glycans contain terminal sialic acid, galactose or N-acetylglucosamine at the non-reducing end.

Sialyltransferase: Sialyltransferases are enzymes that transfer a sialic acid to nascent oligosaccharide. Each sialyltransferase is specific for a particular sugar nucleotide donor substrate. Sialyltransferases add sialic acid to the terminal galactose in glycolipids (gangliosides), or N- or O-linked glycans of glycoproteins. Sialyltransferase is suitable for use in enzymatic conjugation of half life extending moieties to glycans present on the FVIII molecule.

Suitable host cells for producing the FVIII variants according to the invention are preferably of mammalian origin in order to ensure that the molecule is properly processed during folding and post-translational modification, e.g. glycosylation and sulfatation. In practicing the present invention, the cells are mammalian cells, more preferably an established mammalian cell line, including, without limitation, CHO, COS-1, baby hamster kidney (BHK), and HEK293 cell lines. A preferred BHK cell line is the tk-ts13 BHK cell line usually referred to as BHK 570 cells. Other suitable cell lines include, without limitation, Rat Hep I, Rat Hep II, TCMK, NCTC 1469; DUKX cells, and DG44 (CHO cell line). Also useful are 3T3 cells, Namalwa cells, myelomas and fusions of myelomas with other cells. Currently preferred cells are HEK293, COS, Chinese Hamster Ovary (CHO) cells, Baby Hamster Kidney (BHK) and myeloma cells, in particular Chinese Hamster Ovary (CHO) cells. FVIII variants according to the invention may also be produced in transgenic animals (preferably a mammal) or plants (preferably expressed in plant tubers).

Pharmaceutical composition: A pharmaceutical composition is herein preferably meant to encompass compositions comprising Factor VIIII molecules according to the present invention suitable for parenteral administration, such as e.g. ready-to-use sterile aqueous compositions or dry sterile compositions that can be reconstituted in e.g. water or an aqueous buffer. The compositions according to the invention may comprise various pharmaceutically acceptable excipients, stabilizers, etc. Additional ingredients in such compositions may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention. Parenteral administration may be performed by subcutaneous, intramuscular, intraperitoneal or intravenous injection by means of a syringe, optionally a pen-like syringe. Alternatively, parenteral administration can be performed by means of an infusion pump.

Circulatory half life: The term “circulatory half life” as used in connection with the present invention, refers to the circulatory half life measured in vivo. The FVIII variants according to the present invention have a significantly increased circulatory half life as compared with wt FVIII. Preferably the circulatory half life of FVIII variants according to the invention is increased at least about two fold, preferably at least about three fold, more preferably at least about four fold, even more preferably at least about 5, and most preferably at least about 6 fold as compared with wt FVIII. The following assay can be used for measureing the circulatory half life: whole blood clotting time, TEG®, ROTEM®, FVIII:C clot assay, thrombin generation time, chromogenic activity assay, ELISA, etc.

The term “treatment”, as used herein, refers to the medical therapy of any human or other animal subject in need thereof. Said subject is expected to have undergone physical examination by a medical practitioner, who has given a tentative or definitive diagnosis which would indicate that the use of said specific treatment is beneficial to the health of said human or other animal subject. The timing and purpose of said treatment may vary from one individual to another, according to the status quo of the subject's health. Thus, said treatment may be prophylactic, palliative, symptomatic and/or curative.

In a first aspect, the present invention relates to a recombinant FVIII variant having FVIII activity and increased in vitro stability, wherein said FVIII variant is conjugated with a half life extending moiety, and wherein amino acid alterations resulting in increased in vitro stability have been introduced into said FVIII variant. FVIII variants may thus comprise one, two, three, four, five, six, seven, eight, nine, or ten amino acid alterations resulting in increased in vitro stability.

In one embodiment of the present invention, said FVIII variant comprises a disulfide bridge. In another embodiment, said variant comprises two disulfide bridges. In a third embodiment, said variant comprises three disulfide bridges.

In another embodiment of the present invention, said FVIII variant comprises at least one disulfide bridge covalently linking two domains of the FVIII variant. In another embodiment, said FVIII variant comprises at least one disulfide bridge covalently linking the A1 domain to the A2 domain. In another embodiment, said FVIII variant comprises at least one disulfide bridge covalently linking the A2 domain to the A3 domain. In another embodiment, said FVIII variant comprises at least one disulfide bridge covalently linking the A3 domain to the C1 domain. In another embodiment, said FVIII variant comprises two disulfide bridges covalently linking the A1 domain to (i) the A2 and A3 domains, or (ii) the A2 and C2 domains or (ii) the A3 and C2 domains. In another embodiment said FVIII variant comprises at least one disulfide bridge covalently linking the heavy chain to the light chain. In another embodiment, said FVIII variant comprises at least one pair of cysteine residues located at positions selected from a modified computational procedure which follows the rational design procedure of disulfide bonds as described by Dombkowski [Bioinformatics (2003) 19: 1852-3]. The procedure is modified to account for the uncertainty of low resolution x-ray crystallographic structures and might in the case of high resolution structures model the inherent protein plasticity and flexibility. Hence, the new procedure accepts larger tolerances on the bond angles as well on torsion angles describing the geometry of the disulfide bridge.

In another embodiment, said FVIII variant comprises at least one pair of cysteine residues located at positions selected from the group consisting of Gly102-Ala1974, Tyr105-Gly1960, Ser149-Glu1969, Pro264-Gln 645, Ser268-Phe673, Asn280-S524, His281-Asp525, Arg 282-Thr 522, Ser285-Phe673, Glu287-Phe673, His311-Phe 673, Ile 312-Pro 672, Ser 313-Ala 644, Ser313-Gln645, Ser 314-Ala 644, Ser 314-Gln 645, Ser314-Thr646, Asp647-Asn1950, Phe648-Tyr1979, Leu649-Gly1981, Ser650-Gly1981, Gly655-Ala1800, Tyr656-Ser1791, Thr657-Ser1788, Met 662-Lys1827, Met 662-Asp1828, Val 663-Glu1829, Tyr 664-Thr 1826, Y664-Lys1967, Asp666-Ser1788, Thr667-Gly1981, Thr667-Ser1788, Leu668-Ser1788, Gly686-Ser1791, Thr669-Tyr1979, Thr669-Val1982, Phe671-Tyr1979, Gly686-Arg1803, His693-Gly1981, Asn 694-Pro 1980, Asn694-Asn1950, Ser 695-Glu 1844 and Asp696-Asn1950 (positions in SEQ ID NO: 1).

In another embodiment of the present invention, said FVIII variant comprises at least one intra domain disulfide bridge wthin A1, A2 or A3 which contribute to the in vitro stability of the FVIII variant. In another embodiment, said FVIII variant comprises at least one pair of cysteine residues located at positions selected from the group consisting of: Ser13-Lys47, Lys48-Gly171, Val80-Gly145, Gly102-Tyr156, Leu277-Gln297, Lys380-Asp459, Ser650-His693, Ser654-Trp688, Thr1695-Asn1770, Lys1845-Lys1887, Ala1877-Tyr1943 and Ser1946-Leu1978 (positions in SEQ ID NO: 1).

In another embodiment, said FVIII variant according to the invention comprises amino acid substitutions with hydrophobic amino acid residues, wherein the introduced hydrophobic amino acid residues increase the hydrophobic interactions and the in vitro stability of the FVIII variant. In another embodiment, said FVIII variant comprises one or more of the following mutations: Met147Leu, Leu152Pro, Ser313Pro, Leu377Phe, Met539Pro, Thr646Pro, Met662Leu, Cys692Ser, Met1973Leu, and Glu1793Pro (positions in SEQ ID NO: 1).

In another embodiment, said FVIII variant according to the invention comprises amino acid substitutions with altered charges, and wherein the introduced charged residues increase the electrostatic interactions and the in vitro stability of the FVIII variant. In another embodiment, said FVIII variant comprises one, two or more of the following mutations: Gln316Lys, Gln316Lys/Met539Pro, Gln316His, Glu287Ala/Glu676Ala, Asp666Asn, Asp666Val, Arg279Ala/Lys1967Ala, Arg279Gln/Lys1967Gln, Glu287Val, Glu676Val, Glu287Val/Glu676Val, Asp519Ala/Glu665Ala, Asp519Ala/Glu665Val, Asp519Ala/Glu 1984Ala, Asp519Ala/Glu1984Val, Asp519Val/Glu665Val, Asp519Val/Glu1984Ala, Asp519Val/Glu1984Val, Glu665Ala/Glu1984Ala, Glu665Ala/Glu1984Val, Glu665Val/Glu 1984Ala, Glu665Val/Glu1984Val, Asp519Ala/Glu665Val/Glu1984Ala, Asp519Val/Glu665Val/Glu1984Ala, Asp519Val/Glu665Val/Glu1984Val, Asp519Ala, Asp519Val, Asp525Glu/Asp605Glu, Arg489Gly/Asp525Glu/Asp605Glu, Glu665Ala, Glu665Val, Glu1984Ala and Glu1984Val (positions in SEQ ID NO: 1).

In another embodiment, said FVIII variant according to the invention comprises amino acid substitutions which stabilise the binding of metal ions bound to FVIII, e.g. Cu or Zn, either directly or via elimination of oxidation sensitive Methionine residues, and wherein these changes contribute to increasing the in vitro stability of the FVIII variant. In another embodiment, said FVIII variant comprises one or more of the following mutations: Met320Gln, Met320Gln/Met2010Gln, Met2010Gln, Leu649His, Phe697His, Leu649His/Phe697His, Gly2003Ser and Ser313Gly (positions in SEQ ID NO: 1).

In another embodiment, said FVIII variant according to the invention is a B domain truncated variant. In another embodiment, said FVIII variant comprises a half life extending moiety linked to an O-glycan situated in a truncated B-domain, and wherein said moiety is removed upon activation of said FVIII variant. If this variant does not comprise any other half life extending moieties, the activated FVIII variant will thus have a structure that is highly similar to the wt activated FVIII protein. In a preferred embodiment, the sequence of the B domain is as set forth in SEQ ID NO: 2.

In another embodiment, said FVIII variant comprises a half life extending moiety linked to a selectively introduced free cysteine. In another embodiment, said FVIII variant comprises a half life extending moiety linked to a selectively introduced free cysteine and wherein said half life extending moiety is removed upon activation of said FVIII variant. If this variant does not comprise any other side groups, the activated FVIII variant will thus have a structure that is highly similar to the wt activated FVIII protein.

In another embodiment, said FVIII variant according to the invention comprises at least one half life extending moiety selected from the group consisting of a hydrophilic polymer, a PEG group, an antibody (or an antigen binding fragment thereof), an Fc domain, albumin, a polypeptide, and a fatty acid or a fatty acid derivative/an albumin binder. In another embodiment, the half life extending moiety is in the form of a fusion partner fused to said FVIII variant, such as e.g. a FVIII/Fc domain fusion protein, an antibody/FVIII fusion protein, an albumin/FVIII fusion protein or a transferrin/FVIII fusion protein. In another embodiment, the antibody (or antigen binding fragment thereof) is a platelet specific antibody such as e.g. a GPIIb/IIIa specific antibody.

In one embodiment of the invention, the fusion partner is replacing the A3-domain of the FVIII molecule. In another embodiment, the fusion partner is inserted into the B-domain of Factor VIII and the B domain is optionally a truncated B-domain. In another embodiment, the fusion partner is inserted in the N-terminal end of the C2 domain of Factor FVIII.

In one embodiment, said FVIII variant according to the invention has reduced vWF binding capacity.

In one embodiment, the FVIII variant according to the invention comprises the amino acid sequence according to SEQ ID NO: 3 (M662C+D1828C). Preferably, the FVIII variant comprising the amino acid sequence according to SEQ ID NO: 3 (M662C+D1828C) is enzymatically conjugated with a PEG molecule or an albumin binder attached to the O-glycan situated in the truncated B-domain. Preferably, a PEG molecule has a size of about 40 kDa. FVIII variants according to the invention conjugated with a half life extending moiety attached to an O-glycan situated in a B-domain (that may optionally be truncated) generally have the ability to mimic the structure of the wt activated FVIII molecule as the side group in the B domain is removed upon activation of said FVIII variant.

In one embodiment, the FVIII variant according to the invention comprises the following substitutions: S149C and E1969C.

In one embodiment, the FVIII variant according to the invention comprises the following substitutions: D666C and S1788C.

In another embodiment, the FVIII variant according to the invention comprises an amino acid substitution of the N1950 position, wherein said substitution is selected from the group consisting of: N1950Q, N1950F, and N1950I. The substitution is preferably N1950Q or N1950I.

In another embodiment, the FVIII variant according to the invention comprises the following substitutions: D519V and E1984A.

Another aspect relates to a DNA molecule encoding any one of the FVIII variants according to the invention. Another aspect relates to vectors and host cells comprising DNA molecules according to the invention. Another aspect thus relates to methods of producing the FVIII variants according to the invention. Such methods comprise incubating a host cell comprising a DNA molecule encoding a FVIII variant according to the invention under suitable conditions, isolating said FVIII variant and optionally conjugating the FVIII variant with a side group.

Another aspect relates to a pharmaceutical composition comprising the FVIII variant according to the invention, optionally with one or more pharmaceutically acceptable excipients. Preferably, this formulation is a parenteral formulation intended for IV administration. The formulation may be in the form of one container comprising the FVIII variant according to the invention in a lyophilized form and optionally one container containing an aqueous solvent, wherein the lyophilized fraction is dissolved in an aqueous fraction prior to administration.

Another aspect relates to use of a FVIII variant according to the invention for treatment of heamophilia.

Another aspect relates to a method of treatment of haemophilia comprising administering to a person in need thereof a therapeutically efficient amount of a FVIII variant according to the invention.

A final aspect relates to use of a FVIII variant according to the invention for treatment of haemophilia optionally in combination with one or more other drugs used in the treatment of haemophilia (e.g. an inhibitor of a fibrinolytic agent).

### EXAMPLES

As used herein, “N8” and “F8-500” refer to the amino acid sequence of the B-domain truncated FVIII variant previously disclosed in the examples in WO09108806. The “N8”/“F8-500” variant has a B domain with the sequence as set forth in SEQ ID NO: 2 and the activated version of this molecule is essentially identical to endogenous activated FVIII. Specific mutants of this molecule are denoted “F8-500” followed by the specific amino acid substitution according to the numbering of SEQ ID: 1. Some variants of this molecule may furthermore be conjugated to a half life extending moiety, preferably at the O-glycan positioned in the truncated B domain. If e.g. a PEG moiety of 40 kDa is attached, to the O-glycan, the molecule will be named e.g.: “40K-PEG-[O]— . . . .”

### Example 1

### Production of Recombinant B Domain Truncated O-Glycosylated Factor VIII and Variants Thereof, e.g., Factor VIII (M662C-D1828C) or Factor VIII (D519V-E1984A)

Cell Line and Culture Process

Using Factor VIII cDNA, a mammalian expression plasmid was constructed. The plasmids encodes a B-domain deleted Factor VIII comprising the Y1680F mutation, the Factor VIII heavy chain comprising amino acid 1-740 of full length human Factor VIII, and Factor VIII light chain comprising amino acid 1649-2332 of full length human Factor VIII. The heavy and light chain sequences are connected by a 21 amino acid linker (SFSQNSRHPSQNPPVLKRHQR—SEQ ID NO: 2) comprising the sequence of amino acid 741-750 and 1638-1648 of full length human Factor VIII. The Factor VIII amino acid sequence encoded by this plasmid is as set forth in SEQ ID NO: 3 (M662C-D1828C):

(FVIII M662C + D1828C)
SEQ ID NO: 3
ATRRYYLGAVELSWDYMQSDLGELPVDARFPPRVPKSFPFNTSVVYKKTL
FVEFTDHLFNIAKPRPPVVMGLLGPTIQAEVYDTVVITLKNMASHPVSLH
AVGVSYWKASEGAEYDDQTSQREKEDDKVFPGGSHTYVWQVLKENGPMAS
DPLCLTYSYLSHVDLVKDLNSGLIGALLVCREGSLAKEKTQTLHKFILLF
AVFDEGKSWHSETKNSLMQDRDAASARAWPKMHTVNGYVNRSLPGLIGCH
RKSVYWHVIGMGTTPEVHSIFLEGHTFLVRNHRQASLEISPITFLTAQTL
LMDLGQFLLFCHISSHQHDGMEAYVKVDSCPEEPQLRMKNNEEAEDYDDD
LTDSEMDVVRFDDDNSPSFIQIRSVAKKHPKTVVVHYIAAEEEDWDYAPL
VLAPDDRSYKSQYLNNGPQRIGRKYKKVRFMAYTDETFKTREAIQHESGI
LGPLLYGEVGDTLLIIFKNQASRPYNIYPHGITDVRPLYSRRLPKGVKHL
KDFPILPGEIFKYKWTVTVEDGPTKSDPRCLTRYYSSFVNMERDLASGLI
GPLLICYKESVDQRGNQIMSDKRNVILFSVFDENRSVVYLTENIQRFLPN
PAGVQLEDPEFQASNIMHSINGYVFDSLQLSVCLHEVAYVVYILSIGAQT
DFLSVFFSGYTFKHKCVYEDTLTLFPFSGETVFMSMENPGLWILGCHNSD
FRNRGMTALLKVSSCDKNTGDYYEDSYEDISAYLLSKNNAIEPRSFSQNS
RHPSQNPPVLKRHQREITRTTLQSDQEEIDYDDTISVEMKKEDFDIYDED
ENQSPRSFQKKTRHYFIAAVERLWDYGMSSSPHVLRNRAQSGSVPQFKKV
VFQEFTDGSFTQPLYRGELNEHLGLLGPYIRAEVEDNIMVTFRNQASRPY
SFYSSLISYEEDQRQGAEPRKNFVKPNETKTYFWKVQHHMAPTKCEFDCK
AWAYFSDVDLEKDVHSGLIGPLLVCHTNTLNPAHGRQVTVQEFALFFTIF
DETKSVVYFTENMERNCRAPCNIQMEDPTFKENYRFHAINGYIMDTLPGL
VMAQDQRIRVVYLLSMGSNENIHSIHFSGHVFTVRKKEEYKMALYNLYPG
VFETVEMLPSKAGIWRVECLIGEHLHAGMSTLFLVYSNKCQTPLGMASGH
IRDFQITASGQYGQWAPKLARLHYSGSINAWSTKEPFSWIKVDLLAPMII
HGIKTQGARQKFSSLYISQFIIMYSLDGKKWQTYRGNSTGTLMVFFGNVD
SSGIKHNIFNPPIIARYIRLHPTHYSIRSTLRMELMGCDLNSCSMPLGME
SKAISDAQITASSYFTNMFATWSPSKARLHLQGRSNAWRPQVNNPKEWLQ
VDFQKTMKVTGVTTQGVKSLLTSMYVKEFLISSSQDGHQWTLFFQNGKVK
VFQGNQDSFTPVVNSLDPPLLTRYLRIHPQSVVVHQIALRMEVLGCEAQD
LY

Chinese hamster ovary (CHO) cells were transfected with the plasmid and selected with the dihydrofolate reductase system eventually leading to a clonal suspension producer cell cultivated in animal component-free medium.

The first step in the process is the inoculation of a cell vial, from a working cell bank vial, into a chemically defined and animal component free growth medium. Initially after thawing, the cells are incubated in a T-flask. One or two days after thawing, the cells are transferred to a shaker flask, and the culture volume is expanded by successive dilutions in order to keep the cell density between 0.2-3.0×10⁶ cells/ml. The next step is the transfer of the shaker flask culture into seed bioreactors. The culture volume is here further expanded before the final transfer to the production bioreactor. The same chemically defined and animal component free medium is used for all the inoculum expansion steps. After transfer to the production bioreactor, the medium is supplemented with components that increase the product concentration. In the production bioreactor the cells are cultured in a repeated batch process with a cycle time of three days. At harvest, 80-90% of the culture volume is transferred to a harvest tank. The remaining culture fluid is then diluted with fresh medium, in order to obtain the initial cell density, and then a new growth period is initiated. The harvest batch is clarified by centrifugation and filtration and transferred to a holding tank before initiation of the purification process. A buffer is added to the cell free harvest in the holding tank to stabilise pH.

By the end of the production run, cells are collected and frozen, in order to make an end of production cell bank. This cell bank is tested for mycoplasma, sterility and viral contamination.

Purification

For the isolation of B-domain-deleted Factor VIII (M662C-D1828C) from cell culture media a four step purification procedure was used including a concentration step on a Capto MMC column, an immunoabsorbent chromatography step, an anionic exchange chromatography and finally a gelfiltration step. Typically the following procedure was used: 11 litre of sterile filtered medium was pumped onto at column (1.6×12 cm) of Capto MMC (GE Healthcare, Sweden) equilibrated in buffer A: 20 mM imidazole, 10 mM CaCl₂, 50 mM NaCl, 0.02% Tween 80, pH=7.5 at a flow of 15 ml/min. The column was washed with 75 ml of buffer A followed by wash with 75 ml of buffer A containing 1.5 M NaCl. The protein was eluted with 20 mM imidazole, 10 mM CaCl₂, 0.02% Tween 80, 2.5 M NaCl, 8 M ethyleneglycol, pH=7.5 at a flow of 1 ml/min. Fractions of 8 ml were collected and assayed for Factor VIII activity (FVIII:C) in a chromogenic assay (see example 3). Factor VIII containing fractions were pooled and normally a pool volume of around 50 ml was obtained.

A monoclonal antibody against Factor VIII has been developed (Kjalke Eur J Biochem 234 773, Hansen, J Thromb Haemost 2009; 7, Supplement 2: abstract no. PP-WE-572). By further epitope mapping (results not shown) this antibody, F25, was found to recognise the far C-terminal sequence of the heavy chain from amino acid residue 725 to 740. The F25 antibody was coupled to NHS-activated Sepharose 4 FF (GE Healthcare, Bio-Sciences AB, Uppsala, Sweden) at a density of 2.4 mg per ml of gel essentially as described by the manufacturer. The pool from the previous step was diluted 10 times with 20 mM imidazole, 10 mM CaCl₂, 0.02% Tween 80, pH=7.3 and applied to the F25 Sepharose column (1.6×9.5 cm) equilibrated with 20 mM imidazole, 10 mM CaCl₂, 150 mM NaCl, 0.02% Tween 80, 1 M glycerol pH=7.3 at a flow of 0.5 ml/min. The column was washed with equilibration buffer until the UV signal was constant and then with 20 mM imidazole, 10 mM CaCl₂, 0.65 M NaCl, pH=7.3 until the UV signal was constant again. Factor VIII was eluted with 20 mM imidazole, 10 mM CaCl₂, 0.02% Tween 80, 2.5 M NaCl, 50% ethyleneglycol, pH=7.3 at a flow of 1 ml/min. Fractions of 1 ml were collected and assayed for Factor VIII:C (see example 3). Factor VIII containing fractions were pooled and normally a pool volume of around 25 ml was obtained.

A buffer A: 20 mM imidazole, 10 mM CaCl₂, 0.02% Tween 80, 1 M glycerol, pH=7.3 and a buffer B: 20 mM imidazole, 10 mM CaCl₂, 0.02% Tween 80, 1 M glycerol, 1 M NaCl, pH=7.3 was prepared for the ion-exchange step. A column (1×10 cm) of Macro-Prep 25Q Support (Bio-Rad Laboratories, Hercules, Calif., USA) was equilibrated with 85% buffer A/15% Buffer B at a flow of 2 ml/min. The pool from the previous step was diluted 10 times with buffer A and pumped onto the column with a flow of 2 ml/min. The column was washed with 85% buffer A/15% buffer B at a flow of 2 ml/min and Factor VIII was eluted with a linear gradient from 15% buffer B to 70% buffer B over 120 ml at a flow of 2 ml/min. Fractions of 2 ml were collected and assayed for Factor VIII activity (FVIII:C) as described in example 3. Factor VIII containing fractions were pooled and normally a pool volume of around 36 ml was obtained.

The pool from the previous step was applied to a Superdex 200, prep grade (GE Healthcare, Bio-Sciences AB, Uppsala, Sweden) column (2.6×60 cm) equilibrated and eluted at 1 ml/min with 20 mM imidazole, 10 mM CaCl₂, 0.02% Tween 80, 1 M glycerol, 150 mM NaCl, pH=7.3. Fractions of 3 ml were collected and assayed for Factor VIII:C (see example 3). Factor VIII containing fractions were pooled and normally a pool volume of around 57 ml was obtained. The pool containing Factor VIII was store at −80° C.

With the use of the above four-step purification procedure an overall yield of approximately 15% was obtained as judged by FVIII:C and ELISA measurements.

### Example 2

### Procedure for PEGylation of Recombinant 0-Glycosylated Factor VIII

The recombinant Factor VIII molecules obtained in Example 1 are conjugated with polyethylenglycol (PEG) using the following procedure:

For the glycoPEGylation reaction to be efficient a FVIII concentration >5 mg/ml is required. Since FVIII is not normally soluble at the concentration a screening of selected buffer compositions was conducted (see table 1). Based on these considerations a buffer containing 50 mM MES, 50 mM CaCl2, 150 mM NaCl, 20% glycerol, pH 6.0 was found to be a suitable reaction buffer.

Recombinant FVIII which had been purified as described above was concentrated in reaction buffer either by ion exchange on a Poros 50 HQ column using step elution, on a Sartorius Vivaspin (PES) filter, 10 kDa cut-off or on an Amicon 10 kDa MWCO PES filter to a concentration of 6-10 mg/mL. The glycoPEGylation of FVIII was initiated by mixing Factor VIII (BDD) (˜4.7 mg/mL final) with Sialidase (A. urifaciens) (159 mU/mL), CMP-SA-glycerol-PEG-40 kDa (see WO2007/056191) (5 mol.eq.) and MBP-ST3Gal1 (540 mU) (WO 2006102652) in reaction buffer (50 mM MES, 50 mM CaCl2, 150 mM NaCl, 20% glycerol, 0.5 mM antipain, pH 6.0). The reaction mixture was incubated at 32° C. until a conversion yield of ˜20-30% of total.

Following the incubation the sample was diluted with Buffer A (25 mM Tris, 5 mM

CaCl₂, 20 mM NaCl, 20% glycerol, pH 7.5) and applied onto a Source 15Q column (1 cm id×6 cm, 4.7 mL, 1 mL/min, 280 nm). The bound material was washed with Buffer A and eluted using a step gradient with Buffer B (25 mM Tris, 5 mM CaCl₂, 1 M NaCl, 20% glycerol, pH 7.5). GlycoPEGylated Factor VIII-(O)-SA-glycerol-PEG-40 kDa was eluted from the column at ˜25% Buffer B.

In order to block free galactose moieties which had been exposed on the N-glycans during the sialidase treatment the poole fraction of Factor VIII-SA-glycerol-PEG-40 kDa (1.0 mg/mL final) was mixed with CMP-SA (2,000 mol eq) and MBP-SBD-ST3Gal3 (WO 2006102652) (400 mU/mL) in reaction buffer 50 mM MES, 20 mM CaCl2, 150 mM NaCl, 10 mM MnCl2, 20% glycerol, pH 6.0 and incubated at 32° C. for 11 hours.

The resulting capped, glycoPEGylated Factor VIII-SA-glycerol-PEG-40 kDa was seperated from cmp-SA and ST3GalIII by gel-filtration on a Superdex 200 column (10 cm id×300 mm; 280 nm) equilibrated with 50 mM MES, 50 mM CaCl2, 150 mM NaCl, 10% glycerol, pH 6.0; flow rate of 0.25 mL/min. The product Factor VIII-SA-glycerol-PEG-40 kDa elutes at 38 min. The peak fraction was collected, aliquoted and subjected to subsequent analysis.

### Example 3

### O-Glycan 40 kDa-GlycoPEG-BDD-FVIII (M662C-D1828C)

BDD-FVIII (M662C-D1828C—SEQ ID NO: 3) (5.32 mg, 4.4 milligram/ml) in a buffer consisting of: imidazol (20 mM), calcium chloride (10 mM), Tween 80 (0.02%), sodium chloride (500 mM), and glycerol (1 M) in water (pH 7.3) was thawed.

Sialidase (2.4 U, in 20 microliter buffer) from Arthrobacter ureafaciens, sialyl tranferase (His-ST3Gal-I, 2.5 mg/ml, 6.75 U, 125 microliter, EC 2.4.99.4, WO 2006102652), and cytidine monophospate N-5′-PEG-glycerol-neuraminic acid, CMP-SA-glycerol-PEG-40 kDa (1.9 mM, 41 microliter buffer, 78 nmol; see WO2007/056191) were added. The final volume was 1.5 ml. The resulting mixture was left for 24 hours at 23 degrees Celsius. The mixture was diluted to 20 ml with Buffer A: (Imidazol (20 mM), calcium chloride (10 mM), Tween 80 (0.02%), and glycerol (1 M) in water (pH 7.3)).

The resulting mixture was loaded onto a MonoQ 5/50 GL column (GE Healthcare Bio-Sciences, HiHerød, Denmark). The immobilised material was washed with Buffer A (10 column volumes) after which it was eluded from the column using a gradient of: 0-100% Buffer B (Imidazol (20 mM), calcium chloride (10 mM), Tween 80 (0.02%), sodium chloride (1 M), and glycerol (1 M) in water (pH 7.3)) (10 CV 100% A, 10 CV 0-20% Buffer B, 10 CV 20% Buffer B, 25 CV 20-100% Buffer B, and 5 CV 100% Buffer B).

The collected material was mixed with cytidine monophospate N-5′acetyl-neuraminic acid (53 microgram) and sialyltransferase (MBP-SBD-ST3Gal-III, EC 2.4.99.6, see WO 2006102652). The final volume and concentrations were: 2.56 ml and 0.46 mg/ml (FVIII), 0.132 mg/ml (MBP-SBD-ST3Gal-III), and 54 micromolar (cytidine monophospate N-5′acetyl-neuraminic acid), respectively.

The mixture was left for 1 hour at 32 degrees Celsius at which time the mixture was diluted to 20 ml with buffer A. The resulting mixture was loaded onto a MonoQ 5/50 GL column (GE Healthcare Bio-Sciences). The immobilised material was washed with Buffer A after which it was eluded from the column using a gradient of 0-100% (10 CV 100% A, 10 CV 0-20% Buffer B, 10 CV 20% Buffer B, 25 CV 20-100% Buffer B, and 5 CV 100% Buffer B). The protein content in the isolated fractions was evaluated using SDS-PAGE gels (Invitrogen, 7% Tris-Acetate, NuPAGE Tris-Acetate running buffer, 70 minutes, 150 V, non-reduced conditions).

The selected fractions were pooled and concentrated using an Amicon Ultra Centrifuge Tube (Millipore, cut-off: 50 kDa). The volume after concentration was 0.5 ml. The resulting solution was loaded onto a Superose 6 10/300 GL column (GE Healthcare Bio-Sciences, Hillerød, Denmark; column volume 24 ml) that had been pre-equilibrated in a buffer consisting of: Histidine (1.5 g/l), calcium chloride (250 mg/l), Tween 80 (0.1 g/l), sodium chloride (18 g/l), and sucrose (3 g/l) in water (pH 7.0). Using the mentioned buffer and a flow of 0.6 ml/min, the components of the mixture were separated into fractions with a size of 1 ml over 1.5 column volume. The selected fractions pooled (0.015 mg/ml, 2 ml).

### Example 4

### FVIII:C Measured in Chromogenic Assay

The FVIII activity (FVIII:C) of the rFVIII compound was evaluated in a chromogenic FVIII assay using Coatest SP reagents (Chromogenix) as follows: rFVIII samples and a FVIII standard (e.g. purified wild-type rFVIII calibrated against the 7th international FVIII standard from NIBSC) were diluted in Coatest assay buffer (50 mM Tris, 150 mM NaCl, 1% BSA, pH 7.3, with preservative). Fifty μl of samples, standards, and buffer negative control were added to 96-well microtiter plates (Nunc) in duplicates. The factor IXa/factor X reagent, the phospholipid reagent and CaCl₂ from the Coatest SP kit were mixed 5:1:3 (vol:vol:vol) and 75 μl of this added to the wells. After 15 min incubation at room temperature 50 μl of the factor Xa substrate S2765/thrombin inhibitor 1-2581 mix was added and the reactions incubated 10 min at room temperature before 25 μl 1 M citric acid, pH 3, was added. The absorbance at 415 nm was measured on a Spectramax microtiter plate reader (Molecular Devices) with absorbance at 620 nm used as reference wavelength. The value for the negative control was subtracted from all samples and a calibration curve prepared by linear regression of the absorbance values plotted vs. FVIII concentration. The specific activity was calculated by dividing the activity of the samples with the protein concentration determined by HPLC. The concentration of the sample was determined by integrating the area under the peak in the chromatogram corresponding to the light chain and compare with the area of the same peak in a parallel analysis of a wild-type unmodified rFVIII, where the concentration was determined by amino acid analyses. The data in table 1 demonstrate that the specific FVIII:C activity was maintained for the O-glycoPEGylated rFVIII compounds.

### Example 5

### FVIII:C Measured in One-Stage Clot Assay

FVIII:C of the rFVIII compounds was further evaluated in a one-stage FVIII clot assay as follows: rFVIII samples and a FVIII standard (e.g. purified wild-type rFVIII calibrated against the 7th international FVIII standard from NIBSC) were diluted in HBS/BSA buffer (20 mM hepes, 150 mM NaCl, pH 7.4 with 1% BSA) to approximately 10 U/ml followed by 10-fold dilution in FVIII-deficient plasma containing VWF (Dade Behring). The samples were subsequently diluted in HBS/BSA buffer. The APTT clot time was measured on an ACL300R or an ACL5000 instrument (Instrumentation Laboratory) using the single factor program. FVIII-deficient plasma with VWF (Dade Behring) was used as assay plasma and SynthASil, (HemosIL™, Instrumentation Laboratory) as aPTT reagent. In the clot instrument, the diluted sample or standard is mixed with FVIII-deficient plasma, aPTT reagents at 37° C. Calcium chloride is assed and time until clot formation is determined by turbidity. The FVIII:C in the sample is calculated based on a standard curve of the clot formation times of the dilutions of the FVIII standard. The data in table 1 demonstrate the ratio between clotting and chromogenic activity.

TABLE 1
Specific chromogenic activity and clotting activity
relative to the chromogenic ctivity.

| GlycoPEGylated | Specific chromogenic | Ratio between clotting and |
|---|---|---|
| N8 compound | activity (IU/mg) | chromogenic activity |
| N8 | 11 819 ± 727 (5) | 1.02 ± 0.12 (3) |
| F8-500-M662C- | 10076 ± 433 (3) | 0.92 ± 0.05 (3) |
| D1828C | | |
| 40K-PEG-[O]-N8 | 9760 ± 886 (8) | 0.78 ± 0.06 (3) |
| 40K-PEG-[O]- | 11722 ± 699 (3) | 0.58 ± 0.05 (3) |
| F8-500-M662C- | | |

### Example 6

### Guanidinium Chloride Accelerated FVIII In Vitro Stability Assay for Screening of FVIII Variants

The FVIII activities (FVIII:C) plus/minus 1M guanidinium chloride on different FVIII variants were evaluated in a chromogenic FVIII assay using Coatest SP reagents (Chromogenix). The generation and expression of the FVIII mutants was carried out as follows: A fragment encoding the cMyc tag was inserted in the C-terminus of the heavy chain in the expression construct encoding FVIII with a 28 amino acid B-domain linker (Thim L et al. Haemophilia 2010; 16: 349-48). The expression level and activity of this FVIII-cMyc2 were similar to untagged FVIII. Additional restriction sites were added to the FVIII-cMyc2 expression construct to ease swapping of domains among variants.

Serum free transfection was performed using HKB11 cells (Cho M-S et al. J Biomed Sci 2002; 9: 631-63) and 293fectin (Invitrogen) following the manufacturer's recommendations. HKB11 suspension cells were grown in commercial Freestyle 293 Expression Medium (Invi-trogen #. 12338-018) supplemented with 50 U mL-1 penicillin and 50 ug mL-1 streptomycin. Cells were grown as suspension cells in shakers and incubated at 37° C. under 5% CO2 and 95% relative humidity. Cells were seeded at a density of 3×105 cells mL-1 and passaged every 3 to 4 days. Viable and total cell concentrations were evaluated by Cedex (Innovatis) analysis using image analysis software for automated cell counting. Viable cells were high-lighted by their ability to exclude the dye trypan blue. Cells were harvested 96 hours after transfection and the cell pellet isolated by gentle centrifugation. Afterwards, the cell pellet was re-suspended in the Freestyle 293 Expression medium containing 0.5 M NaCl. Following gentle centrifugation, the FVIII containing supernatants were harvested and stored at −80° C. until further analysis.

The rFVIII samples and a FVIII standard (human calibration plasma, Chromogenix) were diluted in Coatest assay buffer (50 mM Tris, 150 mM NaCl, 1% BSA, pH 7.3, with preservative). Five μL of samples (100 ng/ml) were mixed with five μL of 2M guanidinium chloride (final: 1M guanidinium chloride) and another sample with five μL Coatest assay buffer (final: 0M guanidinium chloride) and incubated for 1 h at room temperature allowing denaturation of the FVIII variant. 490 μL of Coatest assay buffer was added and the samples were diluted 4-fold. Fifty μl of the pre-diluted samples (100-, 400-, 1600- and 6400-fold), standards and buffer negative control were added to 96-well Spectramax microtiter plates. The factor IXa/factor X reagent, the phospholipid reagent and CaCl₂ from the Coatest SP kit were mixed 5:1:3 (vol:vol:vol) and 75 μL of this added to the wells. After 15 min incubation at room temperature 50 μL of the factor Xa substrate S-2765/thrombin inhibitor 1-2581 mix was added and the reactions incubated 5 min at room temperature before 25 μL 1 M citric acid, pH 3, was added. The absorbance at 405 nm was measured on an Envision plate reader (PerkinElmer) with absorbance at 620 nm used as reference wavelength. The value for the negative control was subtracted from all samples and a calibration curve prepared by linear regression of the absorbance values of the standards plotted vs. FVIII stability. The stability was calculated as a “Ratio” by dividing the activity of the samples incubated with 1M guanidinium chloride with the activity of the samples incubated with 0M guanidinium chloride. The data in the table demonstrate that only the controls and few of the variants are stable in the assay, especially variants with mutations in position 1950.

TABLE 2
Summary of stabilization data from screening assay for various
FVIII variants designed as described herein in order to
improve FVIII in vitro stability.

| Variant | Ratio |
|---|---|
| Screening data | |
| F8-500-H311Q | 0.000 |
| F8-500-H311Y | 0.000 |
| F8-500-H311F | 0.000 |
| F8-500-H311I | 0.000 |
| F8-500-H311L | 0.000 |
| F8-500-I312L | 0.000 |
| F8-500-I312V | 0.000 |
| F8-500-I312T | 0.000 |
| F8-500-S313N | 0.000 |
| F8-500-S313Q | 0.000 |
| F8-500-S313H | 0.000 |
| F8-500-S313P | 0.000 |
| F8-500-S314V | 0.000 |
| F8-500-S314T | 0.000 |
| F8-500-Q316K | 0.000 |
| F8-500-Q316N | 0.000 |
| F8-500-Q316A | 0.000 |
| F8-500-A644V | 0.000 |
| F8-500-A644T | 0.000 |
| F8-500-A644S | 0.000 |
| F8-500-Q645H | 0.000 |
| F8-500-Q645N | 0.000 |
| F8-500-Q645V | 0.000 |
| F8-500-Q645S | 0.000 |
| F8-500-T646N | 0.000 |
| F8-500-T646S | 0.000 |
| F8-500-T646A | 0.000 |
| F8-500-D647K | 0.000 |
| F8-500-D647Q | 0.000 |
| F8-500-D647N | 0.000 |
| F8-500-F648Y | 0.000 |
| F8-500-F648L | 0.000 |
| F8-500-F648I | 0.000 |
| F8-500-L649I | 0.000 |
| F8-500-L649V | 0.000 |
| F8-500-S650T | 0.000 |
| F8-500-S650V | 0.000 |
| F8-500-M1947H | 0.000 |
| F8-500-M1947Q | 0.000 |
| F8-500-M1947F | 0.000 |
| F8-500-M1947L | 0.026 |
| F8-500-S1949K | 0.000 |
| F8-500-S1949H | 0.000 |
| F8-500-S1949Q | 0.000 |
| F8-500-S1949N | 0.000 |
| F8-500-N1950Q | 0.134 |
| F8-500-N1950F | 0.022 |
| F8-500-N1950I | 0.096 |
| F8-500-N1950L | 0.000 |
| F8-500-N1950V | 0.000 |
| F8-500-E1951K | 0.000 |
| F8-500-E1951H | 0.000 |
| F8-500-E1951Q | 0.000 |
| CONTROLS | |
| F8-500 | 0.016 ± 0.016 |
| F8-500-Q316H (Parker & Lollar, 2007) | 0.084 |
| F8-500-M662C + D1828C (Gale et al, 2006) | 0.325 |
| F8-500-D519V-E1984A (Wakabayashi et al. 2009) | 0.241 |

Parker ET and Lollar P. Biochemistry. 2007; 46: 9737-42
Gale AJ, et al. J Thromb Haemost. 2006; 4: 1315-22.
Wakabayashi H et al. J Thromb Haemost. 2009; 7: 438-44

### Example 7

### Decay in Citrate-Stabilized Plasma

FVIII or FVIII variant (10 μl) were added to 90 μl citrate-stabilized haemophilia A plasma (George King Bio-Medical Inc.) to a concentration of 1 IU/ml and incubated at 37° C. for 0, 3, 6, 20, 24, 44 and 48 hours. Samples were subsequently analyzed for FVIII activity in a chromogenic assay: FVIII samples and dilutions of a FVIII standard (e.g. wild-type FVIII calibrated against the 7th international FVIII standard from NIBSC) were diluted in Coatest assay buffer (50 mM Tris, 150 mM NaCl, 1% BSA, pH 7.3, with preservative). Fifty μl of samples, standards, and buffer negative control were added to 96-well microtiter plates (Nunc) in duplicates. The factor IXa/factor X reagent, the phospholipid reagent and CaCl₂ from the Coatest SP kit were mixed 5:1:3 (vol:vol:vol) and 75 μl of this added to the wells. After 15 min incubation at room temperature 50 μl of the factor Xa substrate S-2765/thrombin inhibitor 1-2581 mix was added and the reactions incubated 10 min at room temperature before 25 μl 1 M citric acid, pH 3, was added. The absorbance at 415 nm was measured on a Spectramax microtiter plate reader (Molecular Devices) with absorbance at 620 nm used as reference wavelength. The value for the negative control was subtracted from all samples, and the remaining FVIII activity of the samples calculated based on a standard curve made of dilutions of the calibrated wild type FVIII. The FVIII activity was plotted versus incubation time, and the plasma half-life (t %) calculated using the equation for one phase decay in GraphPad Prism software. The table below shows plasma t ½ of wildtype FVIII and FVIII with the S149C-E1969C substitutions together with FVIII-M662C-D1828C and FVIII-D519V-E1984A previously described in the literature (Gale A J et al., J Thromb Haemost 2006; 4: 1315-22; Wakabayashi H et al., J Thromb Haemost 2009; 7: 438-44). The plasma stability of FVIII-S149C-E1969C was prolonged as compared to wild-type FVIII.

TABLE 3
Stability of FVIII variants in citrate-stabilized heamophilia A plasma.

Plasma stability, t½ (hrs)

| Sample | best fit value | 95% confidence intervals |
|---|---|---|
| wild-type FVIII | 9.8 | (6.6-18.8) |
| FVIII-D519V-E1984A | 72.3 | (51.7-120.1) |
| (Wakabayashi H et al) | | |
| FVIII-M662C-D1828C | 56.8 | (42.9-83.7) |
| (Gale et al) | | |
| FVIII-S149C-E1969C | 33.8 | (23.5-60.4) |

### Example 8

### Decay in Hirudin/TAP Stabilized Plasma

Citrate-stabilized haemophilia A plasma (George King Bio-Medical Inc.) was added hirudin (5.7 μg/ml) and tick anticoagulant protein (TAP, 12.9 μg/ml) and the plasma recalcified by adding calcium chloride to 20 mM. FVIII or variant (10 μl) were added to 90 μl of the hirudin-TAP stabilized plasma to a concentration of 1 IU/ml and incubated at 37° C. for time intervals up to 7 days e.g. 0, 3, 6, 24, 48, 72, 96, 168, 192 and 216 hours. Samples were subsequently analyzed for FVIII activity in a chromogenic assay as described in the example 8. The table below shows plasma t½ of wildtype FVIII and variants including FVIII-M662C-D1828C and FVIII D519V-E1984A previously described in the literature (Gale A J et al., J Thromb Haemost 2006; 4: 1315-22; Wakabayashi H et al., J Thromb Haemost 2009; 7: 438-44). The data shows that the FVIII variant D666C-S1788C with disulphide bridges inserted between the heavy and light chains has enhanced stability in hirudin-TAP stabilized plasma as compared to wild-type FVIII.

TABLE 4
Stability of FVIII variants in recalcified heamophilia A plasma.

Plasma stability, t½ (hrs)

| Sample | best fit value | 95% confidence intervals |
|---|---|---|
| wild-type FVIII | 35.5 | 29.6-44.3 |
| FVIII-D519V-E1984A | 86.0 | 58.9-160 |
| (Wakabayashi H et al) | | |
| FVIII-M662C-D1828C | 148 | 107-238 |
| (Gale et al) | | |
| FVIII-D666C-S1788C | 134 | 93-240 |

### Example 9

### Thrombin Generation

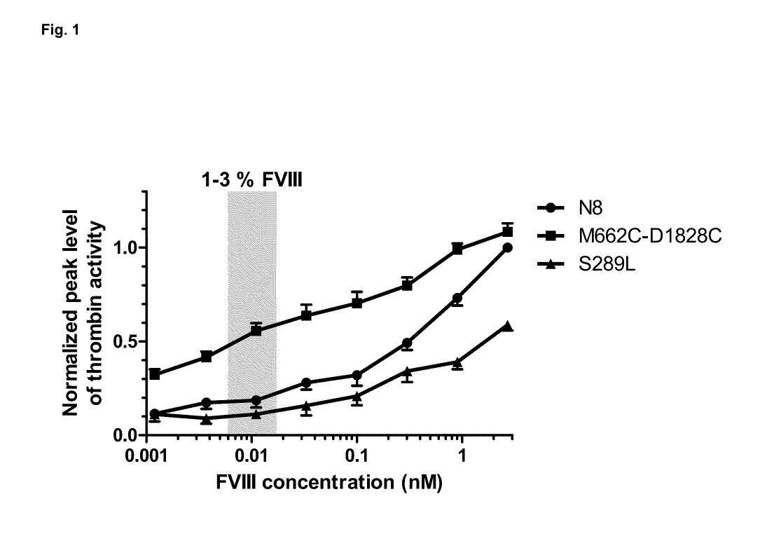

Washed platelets were prepared as described (Lisman T et al. J Thromb Haemost 2005; 3: 742-751) and added to haemophilia A plasma (George King Bio-Medical Inc) to a final density of 150000 platelets/μl. Eighty μl of the platelet-containing plasma was mixed with 5 μl relipidated tissue factor (Innovin, Dade, final dilution 1:50000 corresponding to approx 0.12 μM tissue factor) in microtiter wells and preheated 10 min at 37° C. in a Flouroskan Ascent plate reader (Thermo Electron Corporation). Wild type FVIII or variants (2.7; 0.9, 0.3; 0.1; 0.33; 0.11; 0.0037 and 0.0012 nM final concentration) was added in 15 μl. Fluorogenic substrate (Z-Gly-Gly-Arg-AMC, Bachem, final concentration 417 nM) mixed with CaCl₂ (final concentration 16.7 mM) was added in 20 μl before measuring fluorescence (excitation at 390 nm and emission at 460 nm) continuously for one hour. The fluorescence signal was corrected for α₂-macroglobulinbound thrombin activity and converted to thrombin concentration by use of a calibrator and Thrombinoscope software (Synapse BV) as described (Hemker H C et al. Pathophysiol Haemost Thromb 2003; 33:4-15.). The stabilized FVIII mutant produced more thrombin than wild type FVIII. This was most pronounced at the lower FVIII concentrations analyzed. This is seen when the maximal level of thrombin activity obtained from the Thrombinoscope software is depicted (FIG. 1). The maximal level of thrombin activity obtained with 0.011 nM wild type FVIII and variants are shown in table 4 together with the maximal rate of thrombin generation calculated from the parameters obtained from the Thrombinoscope software, as follows: maximalrate of thrombin generation=maximal level of thrombin activity/(time to peak thrombin activity−lagtime).

TABLE 5
Parameters of thrombin generation obtained by 0.011 nM wild-
type FVIII and variants (mean and standard error of the mean
(SEM) of 5 individual experiments).

| | Rate of thrombin | | Maximal level of | |
|---|---|---|---|---|
| | generation | | thrombin generation | |
| | nM/min | fold-increase* | nM | fold-increase* |
| wild type FVIII | 1.2 ± 0.4 | 1 | 29.7 ± 7.0 | 1 |
| M662C- | 5.4 ± 1.1 | 4.6 | 88.5 ± 4.2 | 3.0 |
| D1828C | | | | |
| S289L | 0.5 ± 0.1 | 0.39 | 18.0 ± 4.7 | 0.61 |

*compared to wild type FVIII

### Example 10

### Pharmacokinetics of rFVIII in FVIII- and VWF-Deficient Mice

The phamacokinetics of rFVIII variants were evaluated in FVIII-deficient mice (FVIII exon 16 knock out (KO) mice with c57bl/6 background, bred at Taconic m&b) or in vWF-deficient mice (vWF exon 4+5 KO mice with c57bl/6 background bred at Charles River, Germany). The vWF-KO mice had 13% of normal FVIII:C, while the FVIII-KO mice had no detectable FVIII:C. A mixture of male and female (approximately 1:1) with an approximate weight of 25 grams and age range of 16-28 weeks were used. The mice received a single i.v. Injections of rFVIII (280 iu/kg) in the tail vein. Blood was taken from the orbital plexus at time points up to 64 hours after dosing using non-coated capillary glass tubes. Three samples were taken from each mouse, and 2 to 4 samples were collected at each time point. Blood was immediately stabilized with sodium citrate and diluted in four volumes FVIII coatest sp buffer (see example 4) before 5 min centrifugation at 4000×g. Plasma obtained from diluted blood was frozen on dry ice and kept at −80° c. The FVIII:C was determined in a chromogenic assay as described in example 4. Pharmacokinetic analysis was carried out by non-compartmental methods (NCA) using winnonlin pro version 4.1 software.

Table 6 show estimates for the pharmacokinetic parameters: the half-life (t½), clearance (cl) and mean residence time (MRT). The data show than the clearance was decreased and the half-life and the mean residence time increased upon PEGylation.

TABLE 6
Pharmacokinetic parameters for FVIII deficient mice.

| | Dose | AUC | T½ | CI | MRT |
|---|---|---|---|---|---|
| Compound | (IU/kg) | (h * IU/mL) | (h) | (mL/h/kg) | (h) |
| N8 | 280 | 26-43 | 7 | 6.5-11 | 10 |
| F8-500-M662C-D1828C | 280 | 30 | 7 | 9.3 | 8.2 |
| 40K-PEG-[O]-N8 | 280 | 73 | 12 | 3.0-4.4 | 17 |
| 40K-PEG-[O]-F8-500-M662C-D1828C | 280 | 124 | 18 | 2.26 | 25.3 |

Matematical models can predict the stability impact on half-life base don plasma half-life in taphirudin stabilized haemophilia plasma.

### Example 11

### Prolonged Haemostatic Effect of Combining PEGylation and FVIII Stabilization in a FeCl₃ Induced Injury Model in Haemophilia A Mice

The duration of action of 40K-PEG-[O]-N8 vs. 40K-PEG-[O]-FVIII (M662C-D1828C) was investigated in a FeCl3 induced injury model in haemophilia A (F8-KO) mice.

Materials and Methods

Mice were anesthetized and placed on a heating pad (37° C.) to maintain body temperature. The carotid artery was exposed and a flow-probe (0.5PSB Nanoprobe) that measures blood flow by ultrasound was placed around the artery. The injury (an iron-mediated chemical oxidation) was induced by applying a filter paper (2×5 mm) briefly soaked in a 10% FeCl3 solution around the exposed carotid artery. The filter paper was removed after 3 min. The artery was then washed three times with 0.9% NaCl and finally Surgilube (an acoustic coupler) was applied in order to displace air in the flow-probe and secure an optimised measurement of the blood flow. Blood flow (ml/min) was recorded for 25 min after removing the FeCl3 saturated filter paper and the time to occlusion was determined by measuring the time (in min) from removal of FeCl3 saturated filter paper until the blood flow was 0 ml/min. If occlusion did not occur after 25 min the occlusion time was reported as 25 min even though no occlusion occurred during the observation period. F8-KO mice (n=6-10) were treated with Advate (280 U/kg), 40K-PEG-[O]-N8 (280 U/kg), or vehicle. The FeCl3 induced injury was made 5 min (acute effect) or 24, 48, 60, and 72 hours after dosing. The blood flow (ml/min) was recorded for 25 min after removal of FeCl3, and subsequently the time to occlusion was determined.

### Results

The FeCl₃ induced injury was made 5 min (acute effect), 72 and 96 hours after dosing 280 IU/kg 40K-PEG-[O]-N8, 280 IU/kg 40K-PEG-[O]-F8 (M662C+D1828C), or vehicle. The blood flow (ml/min) was recorded for 25 min after removal of FeCl₃, and subsequently the time to occlusion was determined (see Table 4). Mean and SEM of 5-8 mice per group are shown. No occlusion occurred in vehicle treated F8-KO mice, whereas occlusion occurred in all mice treated with 40K-PEG-O-N8 and 40K-PEG-[O]-F8 (M662C+D1828C) 5 min after dosing (acute effect) with a mean occlusion time of 3.1±0.5 min and 3.2±0.4 min, respectively. Previous studies in this model reveals that Advate treated F8-KO mice has an occlusion time of 13.0±3.4 min and 15.9±2.9 min after 24 and 48 hours, respectively; however, no occlusions were observed 60 and 72 hours after administration of Advate. In contrast 40K-PEG-[O]-N8 treated F8-KO mice occlusions was observed at both 72 and 96 hours, although with increased average occlusion times (table 5). Interestingly, the stabilized glycoPEGylated FVIII variants shows even more prolonged duration of effect in the FeCl3 induced thrombus formation model compared to glycoPEGylated wild-type FVIII. Thus, when time to occlusion between the different groups was compared using Kruskal-Wallis test including Dunn's post test a statistically significant difference was evident at 96 hours (p<0.05), confirming the added effect of stabilizing the molecule

TABLE 7
Time to occlusion after removal of FeCl₃ saturated filter paper in minutes
(mean ± SEM) n = 5-8

| Time after infusion | 40K-PEG-[O]-F8 | 40K-PEG-[O]-F8 |
|---|---|---|
| (hours) | (wt) | (M662C + D1828C) |
| 0.08 | 3.1 ± 0.5 | 3.2 ± 0.4 |
| 72 | 11.2 ± 3.1 | 6.6 ± 1.0 |
| 96 | 15.9 ± 3.4 | 6.9 ± 1.3 |

1. A recombinant FVIII variant having FVIII activity and increased in vitro stability, wherein said FVIII variant is conjugated with a half life extending moiety, and, wherein amino acid alterations resulting in increased in vitro stability have been introduced into said FVIII variant.

2. The recombinant FVIII variant according to claim 1, wherein said variant comprises a disulfide bridge.

3. The recombinant FVIII variant according to claim 2, wherein said disulfide bridge is covalently linking two domains of the FVIII variant.

4. The recombinant FVIII variant according to claim 2, wherein the disulfide bridge links the heavy chain with the light chain.

5. The recombinant FVIII variant according to claim 1, wherein said FVIII variant comprises amino acid substitutions with hydrophobic amino acid residues, and wherein the introduced hydrophobic amino acid residues increase the hydrophobic interactions and the in vitro stability of the FVIII variant.

6. The recombinant FVIII variant according to claim 1, wherein said variant comprises amino acid substitutions in the form of positively charged and negatively charged amino acid residues, and wherein the introduced charged residues increase the electrostatic interactions and the in vitro stability of the FVIII variant.

7. The recombinant FVIII variant according to claim 1, wherein the variant is a B domain truncated variant.

8. The recombinant FVIII variant according to claim 7, wherein the side group is linked to an O-glycan situated in the truncated B-domain, and wherein said side group is removed upon activation of said FVIII variant.

9. The recombinant FVIII variant according to claim 7, wherein the FVIII, wherein the sequence of the B domain is as set forth in SEQ ID NO: 2.

10. The recombinant FVIII variant according to claim 1, wherein the half life extending moiety is selected from the group consisting of: a hydrophilic polymer, an antibody or an antigen binding fragment thereof, an Fc domain, a polypeptide, and a fatty acid or a fatty acid derivative.

11. The recombinant FVIII variant according to claim 1, wherein said variant comprises the amino acid sequence according to SEQ ID NO: 3.

12. The recombinant FVIII variant according to claim 1, wherein said variant comprises the following substitutions: S149C and E1969C.

13. The recombinant FVIII variant according to claim 1, wherein said variant comprises the following substitutions: D666C and S1788C.

14. A pharmaceutical composition comprising the FVIII variant according to claim 1.

15. A method of treating hemophilia comprising administering the use of a FVIII variant according to claim 1 to a subject in need thereof.
